(19) United States
(12) Patent Application Publication
(10) Pub. No.: US 2021/0322080 A1
(43) Pub. Date: Oct. 21, 2021

(54) HEADLESS SCREW REMOVAL TOOL

(71) Applicant: DEPUY SYNTHES PRODUCTS, INC., Raynham, MA (US)

(72) Inventors: Gregor Spreiter, Zuchwil (CH); Henri Défossez, Neuchatel (CH); Simon Scherrer, Zurich (CH); Markus Buettler, Gunsberg (CH)

(21) Appl. No.: 16/851,076

(22) Filed: Apr. 16, 2020

(51) Int. Cl. A61B 17/92; A61B 17/86

(52) U.S. Cl. CPC A61B 17/92; A61B 17/1615; A61B 17/8645

(57) ABSTRACT

A bone screw removal device includes a shaft extending longitudinally from a proximal end to a distal removal tip. The removal tip includes a first portion at a distal-most end of the removal tip, a second portion extending proximally from the first portion and a third portion extending proximally from the second portion. The first portion is sized and shaped to be received within a retaining recess of a bone screw to clear away tissue growth as the first portion is inserted through a driving recess of a bone screw and into the retaining recess. The second portion is configured to be received within a driving recess. The third portion is configured to clear away tissue growth over a proximal end of the bone screw as the first and second portions are inserted into the retaining recess and the driving recess, respectively.

### BACKGROUND

Bone screws are often used to attach bone fixation devices such as, for example, intramedullary nails and/or bone plates, to bone. In some cases, these bone screws must be removed from the bone after implantation. Bone screws are generally driven into and removed from a bone using a screwdriver and/or an extraction device including a distal end configured to engage, for example, a driving recess at a proximal end of the bone screw so that rotation of the screwdriver correspondingly rotates the bone screw.

In some cases, however, tissue ingrowth over the driving recess makes it difficult for a conventional screwdriver/extraction device to engage the bone screw to remove the bone screw from the bone. Multiple tools may be required to clear the bone prior to extraction, which may be difficult and time consuming. In some cases, a conventional reamer may be used to clear the tissue ingrowth around the screw and access the driving recess. A reamer, however, may leave large holes for the screw, substantially weakening the bone around the screw, and/or result in metal debris.

### SUMMARY

The present disclosure relates a bone screw removal device which includes a shaft extending longitudinally from a proximal end to a distal removal tip. The removal tip includes a first portion at a distal-most end of the removal tip, a second portion extending proximally from the first portion and a third portion extending proximally from the second portion. The first portion is sized and shaped to be received within a retaining recess of a bone screw to clear away tissue growth as the first portion is inserted through a driving recess of a bone screw and into the retaining recess. The second portion is configured to be received within a driving recess. The third portion is configured to clear away tissue growth over a proximal end of the bone screw as the first and second portions are inserted into the retaining recess and the driving recess, respectively.

In an embodiment, the first portion includes cutting flutes extending along a length thereof to clear away tissue growth.

In an embodiment, one of a length of the first portion substantially corresponds to a length of the retaining recess and a length of the second portion substantially corresponds to a length of the driving recess to deteiinine a maximum depth of insertion of the removal tip relative to the bone screw.

In an embodiment, the first portion includes a blunt end.

In an embodiment, the second portion is sized and shaped to be received within the driving recess of the bone screw and includes cutting flutes extending along a length thereof to clear away tissue growth within the driving recess.

In an embodiment, the second portion is substantially conically shaped, increasing in diameter from a distal end to a proximal end thereof.

In an embodiment, the second portion includes a reverse threading extending therealong.

In an embodiment, the second portion includes a distal portion configured to be received within the retaining recess and a proximal portion configured to be received within the driving recess.

In an embodiment, the second portion further includes a step extending about the second portion between the distal and proximal portions thereof.

In an embodiment, the third portion includes cutting flutes extending along a length of an exterior surface thereof to clear away tissue ingrowth extending over the proximal end of the hone screw.

In an embodiment, a diameter of the third portion corresponds to a diameter of the bone screw.

The present disclosure also relates to a system for removing a bone screw. The system includes a bone screw extending from a proximal end to a distal end and including a driving recess extending distally into the proximal end and a retaining recess extending distally from the driving recess; and a removal device for clearing away tissue growth around a previously implanted bone screw. The removal device includes a shaft extending longitudinally from a proximal end to a distal removal tip. The removal tip includes a first portion at a distal-most end of the removal tip, a second portion extending proximally from the first portion and a third portion extending proximally from the second portion. The first portion is sized and shaped to be received within a retaining recess of the bone screw to clear away tissue growth therewithin. The second portion is configured to be received within the driving recess. The third portion is configured to clear away tissue growth over the proximal end of the bone screw.

In an embodiment, the first and third portions includes cutting flutes for clearing away the tissue growth as the removal device is rotated in a removal direction about a longitudinal axis thereof as the removal tip is inserted into the proximal end of the bone screw.

In an embodiment, one of a length of the first portion substantially corresponds to a length of the retaining recess and a length of the second portion substantially corresponds to a length of the driving recess to deteiiiiine a maximum depth of insertion of the removal tip relative to the bone screw.

In an embodiment, the second portion is sized and shaped to be received within the driving recess of the bone screw and includes cutting flutes extending along a length thereof to clear away tissue growth within the driving recess as the removal device is rotated in a removal direction about a longitudinal axis thereof.

In an embodiment, the second portion is substantially conically shaped, the second portion increasing in diameter from a distal end to a proximal end thereof

In an embodiment, the second portion includes a distal portion configured to be received within the retaining recess and a proximal portion configured to be received within the driving recess.

In an embodiment, the second portion further includes a step extending about the second portion between the distal and proximal portions thereof, the step configured to engage a distal end of the driving recess to prevent further distal movement of the removal device relative to the bone screw.

In an embodiment, the second portion includes a reverse threading extending therealong so that, when the removal device is rotated in a removal direction about the longitudinal axis thereof, a portion of the reverse threading engages the driving recess so that further rotation of the removal device in the removal direction extracts the bone screw from the bone.

In an embodiment, a diameter of the third portion corresponds to a diameter of the bone screw.

In addition, the present disclosure relates to a method for removing a previously implanted bone screw. The method includes clearing tissue growth surrounding a previously implanted bone screw by inserting a removal tip of a removal device into a proximal end of the bone screw, the removal tip including a first portion sized and shaped to be received within a retaining recess of a bone screw and configured to clear away tissue growth therewithin, a second portion extending proximally from the first portion to be received within a driving recess of a bone screw and a third portion configured to clear tissue extending over the proximal end of the bone screw as the first and second portions are inserted into the retaining recess and the driving recess, respectively; and extracting the bone screw via a path cleared via the third portion of the removal tip.

In an embodiment, the clearing the tissue growth includes rotating a shaft of the removal tool in a removal direction about a longitudinal axis thereof so that cutting flutes extending along the first and third portions clear away the tissue growth.

In an embodiment, the second portion of the removal tip includes cutting flutes extending therealong so that, when the removal device is rotated in the removal direction the cutting flutes of the second portion clear away tissue within the driving recess.

In an embodiment, the second portion is substantially conically shaped, increasing in diameter from a distal end to a proximal end thereof so that, the second portion is received within the driving recess and the retaining recess via a path cleared via the first portion.

In an embodiment, the second portion includes a distal portion configured to be received within the retaining recess and a proximal portion configured to be received within the driving recess.

In an embodiment, the removal tip is inserted into the proximal end of the bone screw until a step extending about the second portion between the proximal and distal portions of the second portion abuts a distal end of the driving recess.

In an embodiment, extracting the bone screw includes rotating the removal device about the longitudinal axis thereof in the removal direction so that a reverse threading extending about and along the second portion engages the driving recess.

In an embodiment, further rotation of the removal device in the removal direction extracts the bone screw from the bone.

### BRIEF DESCRIPTION

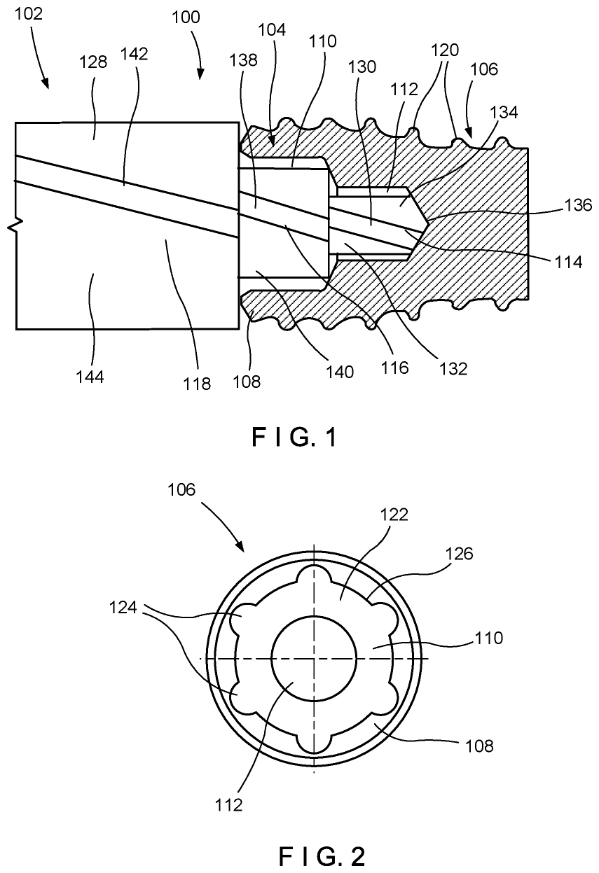

FIG. 1 shows a longitudinal cross-sectional view of a system according to an exemplary embodiment of the present disclosure;

FIG. 2 shows a plan view of a bone screw according to the system of FIG. 1;

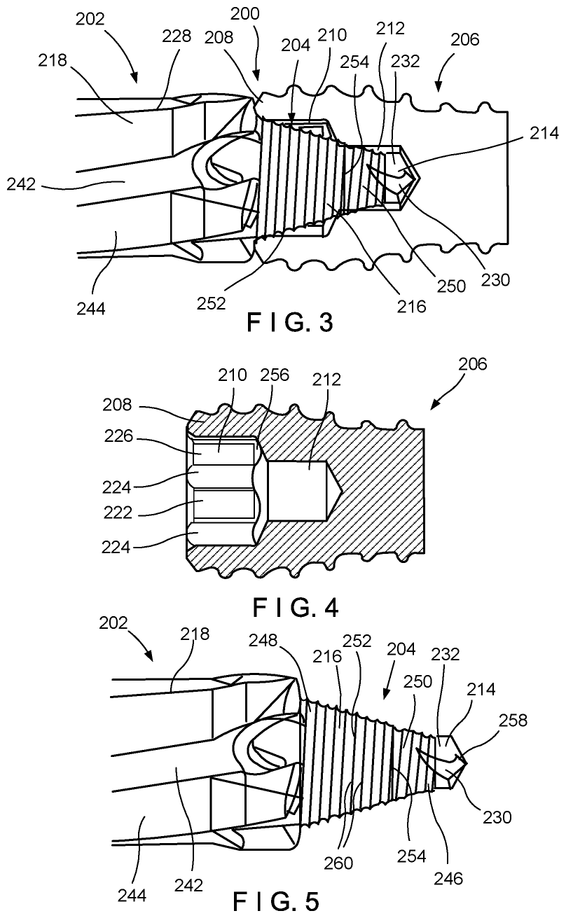

FIG. 3 shows a partial longitudinal cross-sectional view of a system according to another exemplary embodiment of the present disclosure;

FIG. 4 shows a longitudinal cross-sectional view of a screw according to the system of FIG. 2; and

FIG. 5 shows a side view of an extraction device according to the system of FIG. 2.

### DETAILED DESCRIPTION

The present disclosure may be understood with reference to the following description and the appended drawings, wherein like elements are referred to with the same reference numerals. The present embodiments relate to the treatment of bone and, in particular, relate to screw extraction devices for removing bone screws from bone after implantation. Exemplary embodiments describe a removal device having a distal tip including different portions, each of which is configured to clear and/or access a different portion of the bone screw so that the tissue ingrowth surrounding the screw may be cleared in a single step rather than requiring multiple tools, which may be time consuming, and/or which may generate metal debris as when using a conventional reamer. Thus, the removal devices of the exemplary embodiments reduce the number of surgical steps required, reduce a time required in the operating room and reduce metal debris.

It will be understood by those of skill in the art that although the exemplary embodiments are specifically shown and described with respect to a headless screw, the removal devices of the present disclosure may be similarly used for extracting any of a variety of bone screws in any of a number of different types of bone fixation systems. It should be noted that the terms proximal and distal, as used herein, are intended to refer to a direction toward (proximal) and away from (distal), respectively, a user of the device.

As shown n FIGS. 1-2, a screw removal system 100 comprises a removal device 102 including a removal tip 104 configured to clear and access a driving recess 110 and a retaining recess 112 at a proximal end 108 of a bone screw 106. In this embodiment, the extraction tip 104 includes three portions—a first portion 114 for accessing and clearing bony ingrowth within the retaining recess 112, a second portion 116 for clearing bony ingrowth within the driving recess 110, and a third portion 118 for clearing bony growth extending over the proximal end 108 of the bone screw 106. As will be understood by those of skill in the art, once the removal tip 104 has cleared the driving recess 110, as will be described in further detail below, an extraction device may access and engage the driving recess 110 to extract the bone screw from the bone.

The bone screw 106 may have any of a variety of configurations so long as the bone screw 106 extends from the proximal end 108 to a distal end and includes a threading 120 extending therealong so that the bone screw 106 may be rotationally driven into and engaged with bone. In one embodiment the bone screw 106 is a headless screw so that the threading 120 extends along substantially an entire length of the screw 106. The driving recess 110 extends into the proximal end 108 of the bone screw 106, and is engageable with one of a driving device (e.g., screwdriver), as will be understood by those of skill of the art, include features for engaging the driving recess 110 so that rotation of the driving device about a longitudinal axis thereof correspondingly rotates the bone screw 106 so that the bone screw 106 may be driven into and/or extracted from the bone, respectively. The retaining recess 112 extends distally from the driving recess 110 and is configured to engage a retaining feature of the driving device.

In one embodiment, as shown in FIG. 2, the driving recess 110 includes a substantially circular central portion 122 along with a plurality of notches 124 formed along a surface 126 of the substantially circular central portion 122 so that the notches 124 extend radially outward from a longitudinal axis of the bone screw 106. In one embodiment, the driving recess 110 includes six notches 124 which are equally circumferentially spaced from one another. It will be understood by those of skill in the art that the driving recess 110 may include any number of notches 124 in any of a variety of configurations so long as the notches 124 are sized and shaped to engage corresponding portions of a driving device and/or extraction device. It will also be understood by those of skill in the art, that the driving recess 110 may have any of a variety of configurations so long as the driving recess 110 is engageable with a corresponding driving device/extraction device and, in some cases, may not include notches 124. For example, in another embodiment, the driving recess 110 may have a hexagonal cross-section.

The retaining recess 112 extends distally from the driving recess 110 and includes a retaining structure such as, for example, threading, for engaging a retaining portion of a driving device so that the bone screw 106 is prevented from inadvertently disengaging from the driving device as the bone screw 106 is being driven into the bone. The retaining recess 112 has a smaller diameter than the driving recess 110 and, in one embodiment, is coaxial with the central portion 122 of the driving recess 110.

As described above, the removal device 102 may be used to clear tissue surrounding a previously implanted bone screw such as, for example, the bone screw 106, so that the bone screw 106 may be extracted from the bone. In some cases, tissue ingrowth within the driving recess 110 and surrounding the bone screw 106 may make it difficult to extract the bone screw 106 using a convention screwdriver or extraction device. The removal device 102 includes a longitudinally extending shaft 128 extending from a proximal end to the removal tip 104.

First and second portions 114, 116 of the removal tip 104 are configured to be received within the retaining recess 112 and the driving recess 110, respectively, while the third portion 118 is configured to cut away tissue growth proximal to the proximal end 108 of the bone screw 106 to clear a path via which the bone screw 106 may be removed from the bone. In one embodiment, the proximal end (not shown) of the shaft 128 includes a handle portion configured to facilitate gripping thereof by a user (e.g., surgeon) so that the removal device 102 may be rotated about a longitudinal axis of the device 102, as will be described in further detail below, to facilitate insertion of the distal tip into the driving recess 110 and the retaining recess 112.

The first portion 114 is sized, shaped and configured to be received within the retaining recess 112. The first portion 114 is configured to self-align with the retaining recess 112 as it is inserted into the proximal end 108 of the bone screw 106 and includes cutting flutes 130 extending along a length of an exterior surface 132 thereof to cut through any in grown tissue as it is inserted into the retaining recess 112. In one embodiment, a length of the first portion 114 is configured to correspond to a length of the retaining recess 112 so that, when a distal end 134 abuts a distal end 136 of the retaining recess 112, the removal tip 104 is prevented from moving further distally relative to the bone screw 106. In particular, the distal end 134 in this embodiment is blunt and acts as a stop for the removal tip 104 so that second and third portions 116, 118 of the removal tip 104 do not cut or drill into the metal of the bone screw 106.

The second portion 116 extends proximally from the first portion 114 and is sized, shaped and configured to be received within the driving recess 110. In one embodiment, the second portion 116 is sized and shaped to be received within the central portion 122 of the driving recess 110 and includes a length corresponding to a length of the driving recess. The second portion 116 includes cutting flutes 138 extending along the length of an exterior surface 140 thereof to facilitate cutting away of ingrown tissue as the second portion 116 is moved into the driving recess 110. The second portion 116 clears ingrown tissue within the driving recess while also controlling a depth of insertion of the removal tip 104 relative to the bone screw 106.

The third portion 118 extends proximally from the second portion 116 and includes cutting flutes 142 along an exterior surface 144 thereof to facilitate cutting away/clearing of tissue that has grown over the proximal end 108 of the screw 106 to create an exit path through which the screw 106 may be extracted. The third portion 118 of this embodiment has a diameter 118 corresponding to a diameter (although it may be made slightly larger than the diameter of the bone screw 106) so that the exit path is sufficiently large to permit the bone screw 106 be extracted there through. As will be understood by those of skill in the art, headless screws may be inserted beyond or close to an outer bone surface resulting in hone ingrowth over the proximal end 108 of the bone screw 106. In such cases, the third portion 118 clears away the bone ingrowth covering the proximal end 108 of the bone screw 106. As described above, the first and second portions 114, 16 are sized so that, the third portion 118 cannot be drilled distally into any portion of the bone screw 106.

The cutting flutes 130, 138, 142 of each of the first, second and third portions 114, 116, 118, respectively, of the removal tip 104 are configured to cut through the hone and tissue growth as the removal device 102 is rotated about the longitudinal axis thereof Thus, as the removal device 102 is moved distally relative to the bone screw 106 substantially along a longitudinal axis of the bone screw 106 and rotated about the longitudinal axis of the removal device 102, tissue growth around and within the bone screw 106 is cut and cleared as the first and second portions 114, 116 move into the retaining recess 112 and driving recess 110, respectively. The removal device 102 may be rotated about the longitudinal axis in either the clockwise or the counter-clockwise direction, such that the first, second and third portions 114, 116, 118, respectively, of the removal tip 104 may have both a clockwise cutting direction and a counter-clockwise cutting direction.

Once the tissue growth surrounding and within the bone screw 106 has been cleared using the removal device 102, as described above, the driving recess 110 and the retaining recess 112 may receive corresponding portions of an extraction tool so that the bone screw 106 may be extracted from the bone via the path cleared by the third portion 118.

As shown in FIGS. 3-5, a screw removal system 200 according to another exemplary embodiment is substantially similar to the system 100 described above, comprising a removal device 202 including a removal tip 204 configured to clear bone ingrowth proximal to a proximal end 208 of a bone screw 206 and within a driving recess 210 and a retaining recess 212 of the bone screw 206. Similarly to the removal device 102, the removal tip 204 of the removal device 202 includes a first portion 214, a second portion 216 and a third portion 218 each of which is configured to clear hone growth surrounding or within the bone screw 206. As will be described in further detail below, the removal tip 204 of the removal device 202 is configured so that, once the bone ingrowth has been cleared, the removal tip 204 may be used to engage a portion of the driving recess 210 so that the removal device 202 may also be used to extract the bone screw 206 from the bone.

As shown in FIG. 4, the bone screw 206 may be substantially similar to the bone screw 106 including the driving recess 210 extending distally into the proximal end 208 thereof. The retaining recess 212 extends distally from the driving recess 210. In one embodiment, similarly to the bone screw 106, the driving recess 210 may include a central portion 222 with a plurality of notches 224 extending radially outward from a surface 226 thereof. The driving recess 210 may have any of a variety of shapes and sizes so long as the driving recess 210 is configured to engage a corresponding driving portion of a screwdriver/extraction tool. The retaining recess 212 has a diameter smaller than that of the driving recess 210 and, in this embodiment, includes threading extending therealong for engaging threading of a corresponding retaining portion of a screwdriver/extraction tool configured for use therewith. Further, the bone screw 206 has a driving direction (e.g., clockwise) about a longitudinal axis thereof wherein the bone screw 206 is inserted distally into the bone and a removal direction (e.g., counter-clockwise), opposite of the driving direction, wherein the bone screw 206 is removed proximally out of the bone.

As shown in FIG. 5, similarly to the removal device 102, the removal device 202 includes an extending shaft 228 extending longitudinally from a proximal end to the removal tip 204. As described above, the removal tip 204 includes the first portion 214, the second portion 216 and the third portion 218. The first portion 214 extends along a distal-most portion of the removal tip 204 and is sized and shaped to be received within the retaining recess 212. Similarly to the first portion 114, the first portion 214 includes cutting flutes 230 extending along a length of an exterior surface 232 thereof for cutting away bone ingrowth as the first portion 214 is inserted into the proximal end 208 of the bone screw 206 and through the retaining recess 212.

The removal device 202, similarly to the removal device 102, may be rotated about a longitudinal axis of the removal device 202 in either the clockwise or the counter-clockwise direction, such that the first, second and third portions 214, 216, 218, respectively, of the removal tip 204 may have both a clockwise cutting direction and a counter-clockwise cutting direction. In particular, when the removal device 202 is rotated in a removal direction (e.g., counter-clockwise) about the longitudinal axis, as it is inserted into the proximal end 208 of the screw 206, the cutting flutes 230 of the first portion 214 cut away bone ingrowth within the driving recess 210 and the retaining recess 212.

The second portion 216 has a substantially conical shape which increases in diameter from a distal end 246 thereof to a proximal end 248 thereof. A distal portion 250 of the second portion 216 is sized to be received within the retaining recess 212 while a proximal portion 252 of the second portion 216 is configured to be received within the driving recess 210. It will be understood by those of skill in the art that the conical shape of the second portion 216 facilitates insertion of the second portion into the driving recess 210 and the retaining recess 212 via the path cleared by the first portion 214.

In one embodiment, a step 254 or shoulder extends about the second portion 216, between the distal and proximal portions 250, 252 so that, when the step 254 abuts a distal surface 256 of the driving recess 210, the removal device 210 is prevented from moving further distally with respect to the bone screw 206. Thus, in one embodiment, a length of the removal tip 204 from the step 254 to a distal end 258 of the first portion 214 substantially corresponds to a length of the retaining recess 212. A length of the proximal portion 252 of the second portion 216 in this embodiment substantially corresponds to a length of the driving recess 210.

The second portion 216 includes a reverse threading 260 extending along a length thereof. The reverse threading 260 is configured so that, when the removal device 202 is further rotated in the removal direction, a portion of the reverse threading 260 engages the driving recess 210 until the proximal end of the second portion 216 fully engages the driving recess 210. Due to the reverse threading 260, a continuous rotation of the removal device 202 in the removal direction correspondingly rotates the bone screw 206 to extract the bone screw 206 from the bone. In one embodiment, the reverse threading 260 engages the surface 226 of the central portion 222 of the driving recess 210 so that portions thereof extend into the notches 224. Thus, a diameter of the second portion 216 at the proximal end 248 is substantially similar to a diameter of the driving recess 210 defined via the notches 224.

The third portion 218 of the removal tip 204 is substantially similar to the third portion 118 of the removal device 102, extending proximally from the second portion 216. The third portion similarly includes cutting flutes 242 along an exterior surface 244 thereof to facilitate cutting away/clearing of tissue growth over the proximal end 208 of the hone screw 206. The third portion 218 of this embodiment has a diameter corresponding to a diameter of the bone screw 206 so that the third portion 218 clears a path sufficiently large to permit the bone screw 206 to be extracted from the bone.

As described above, to clear bone ingrowth which may impede the extraction of the bone screw 206, the removal tip 204 is inserted in a distal direction into the proximal end 208 of the bone screw 206, rotating the removal device 202 in the removal direction so that the cutting flutes 230, 242 along the first and third portions 214, 218 clear away bone ingrowth within the driving and retaining recesses 210, 212 and proximally of the proximal end 208 of the bone screw 206 as the first portion 214 moves into the retaining recess 212.

The removal tip 204 may be driven distally relative to the bone screw 206 until the step 254 abuts the distal end 256 of the driving recess 210, indicating to a user (e.g., surgeon) that the surrounding tissue has been sufficiently cleared. The user may then rotate the removal device 202 in the removal direction so that the reverse threading 260 along the proximal portion 252 of the second portion 216 engages the driving recess 210. Once engaged, further rotation of the removal device 202 in the removal direction correspondingly rotates the bone screw 206 to extract the bone screw 206 from the bone.

It will be understood by those of skill in the art that modifications and variations may be made in the structure and methodology of the present embodiment, without departing from the spirit or the scope of the embodiments. Thus, it is intended that the present embodiments cover the modifications and variations of these embodiments provided that they come within the scope of the appended claims and their equivalents.

What is claimed is:

1. A bone screw removal device, comprising:
a shaft extending longitudinally from a proximal end to a distal removal tip, the removal tip including a first portion at a distal-most end of the removal tip, a second portion extending proximally from the first portion and a third portion extending proximally from the second portion, the first portion sized and shaped to be received within a retaining recess of a bone screw to clear away tissue growth as the first portion is inserted through a driving recess of a bone screw and into the retaining recess, the second portion configured to be received within a driving recess and the third portion configured to clear away tissue growth over a proximal end of the bone screw as the first and second portions are inserted into the retaining recess and the driving recess, respectively.

2. The device of claim 1, wherein the first portion includes cutting flutes extending along a length thereof to clear away tissue growth.

3. The device of claim 1, wherein one of a length of the first portion substantially corresponds to a length of the retaining recess and a length of the second portion substantially corresponds to a length of the driving recess to determine a maximum depth of insertion of the removal tip relative to the bone screw.

4. The device of claim 1, wherein the first portion includes a blunt end.

5. The device of claim 1, wherein the second portion is sized and shaped to be received within the driving recess of the bone screw and includes cutting flutes extending along a length thereof to clear away tissue growth within the driving recess.

6. The device of claim 1, wherein the second portion is substantially conically shaped, increasing in diameter from a distal end to a proximal end thereof.

7. The device of claim 6, wherein the second portion includes a reverse threading extending therealong.

8. The device of claim 6, wherein the second portion includes a distal portion configured to be received within the retaining recess and a proximal portion configured to be received within the driving recess.

9. The device of claim 7, wherein the second portion further includes a step extending about the second portion between the distal and proximal portions thereof.

10. The device of claim 1, wherein the third portion includes cutting flutes extending along a length of an exterior surface thereof to clear away tissue ingrowth extending over the proximal end of the bone screw.

11. The device of claim 1, wherein a diameter of the third portion corresponds to a diameter of the bone screw.

12. A system for removing a bone screw, comprising:
a bone screw extending from a proximal end to a distal end and including a driving recess extending distally into the proximal end and a retaining recess extending distally from the driving recess; and
a removal device for clearing away tissue growth around a previously implanted bone screw, the removal device including a shaft extending longitudinally from a proximal end to a distal removal tip, the removal tip including a first portion at a distal-most end of the removal tip, a second portion extending proximally from the first portion and a third portion extending proximally from the second portion, the first portion sized and shaped to be received within a retaining recess of the bone screw to clear away tissue growth therewithin, the second portion configured to be received within the driving recess and the third portion configured to clear away tissue growth over the proximal end of the bone screw.

13. The system of claim 12, wherein the first and third portions includes cutting flutes for clearing away the tissue growth as the removal device is rotated in a removal direction about a longitudinal axis thereof as the removal tip is inserted into the proximal end of the bone screw.

14. The system of claim 12, wherein one of a length of the first portion substantially corresponds to a length of the retaining recess and a length of the second portion substantially corresponds to a length of the driving recess to determine a maximum depth of insertion of the removal tip relative to the bone screw.

15. The system of claim 12, wherein the second portion is sized and shaped to be received within the driving recess of the bone screw and includes cutting flutes extending along a length thereof to clear away tissue growth within the driving recess as the removal device is rotated in a removal direction about a longitudinal axis thereof.

16. The system of claim 12, wherein the second portion is substantially conically shaped, the second portion increasing in diameter from a distal end to a proximal end thereof

17. The system of claim 16, wherein the second portion includes a distal portion configured to be received within the retaining recess and a proximal portion configured to be received within the driving recess.

18. The system of claim 17, wherein the second portion further includes a step extending about the second portion between the distal and proximal portions thereof, the step configured to engage a distal end of the driving recess to prevent further distal movement of the removal device relative to the bone screw.

19. The system of claim 17, wherein the second portion includes a reverse threading extending therealong so that, when the removal device is rotated in a removal direction about the longitudinal axis thereof, a portion of the reverse threading engages the driving recess so that further rotation of the removal device in the removal direction extracts the bone screw from the bone.

20. The system of claim 12, wherein a diameter of the third portion corresponds to a diameter of the bone screw.

21. A method for removing a previously implanted bone screw, comprising:
clearing tissue growth surrounding a previously implanted bone screw by inserting a removal tip of a removal device into a proximal end of the bone screw, the removal tip including a first portion sized and shaped to be received within a retaining recess of a bone screw and configured to clear away tissue growth therewithin, a second portion extending proximally from the first portion to be received within a driving recess of a bone screw and a third portion configured to clear tissue extending over the proximal end of the bone screw as the first and second portions are inserted into the retaining recess and the driving recess, respectively; and
extracting the bone screw via a path cleared via the third portion of the removal tip.

22. The method of claim 21, wherein the clearing the tissue growth includes rotating a shaft of the removal tool in a removal direction about a longitudinal axis thereof so that cutting flutes extending along the first and third portions clear away the tissue growth.

23. The method of claim 22, wherein the second portion of the removal tip includes cutting flutes extending therealong so that, when the removal device is rotated in the removal direction the cutting flutes of the second portion clear away tissue within the driving recess.

24. The method of claim 22, wherein the second portion is substantially conically shaped, increasing in diameter from a distal end to a proximal end thereof so that, the second portion is received within the driving recess and the retaining recess via a path cleared via the first portion.

25. The method of claim 24, wherein the second portion includes a distal portion configured to be received within the retaining recess and a proximal portion configured to be received within the driving recess.

26. The method of claim 25, wherein the removal tip is inserted into the proximal end of the bone screw until a step extending about the second portion between the proximal and distal portions of the second portion abuts a distal end of the driving recess.

27. The method of claim 26, wherein extracting the bone screw includes rotating the removal device about the longitudinal axis thereof in the removal direction so that a reverse threading extending about and along the second portion engages the driving recess.

28. The method of claim 27, wherein further rotation of the removal device in the removal direction extracts the bone screw from the bone.
